(19) United States
(12) Patent Application Publication
(10) Pub. No.: US 2016/0169853 A1
(43) Pub. Date: Jun. 16, 2016

(54) METHOD FOR DETERMINING SUITABILITY OF MARINE FUELS

(71) Applicants: Frank P. DiSanzo, Cherry Hill, NJ (US); Heather Hamje, Brussels (BE); David J. Blasetto, Gibbstown, NJ (US)

(72) Inventors: Frank P. DiSanzo, Cherry Hill, NJ (US); Heather Hamje, Brussels (BE); David J. Blasetto, Gibbstown, NJ (US)

(73) Assignee: ExxonMobil Research and Engineering Company, Annandale, NJ (US)

(21) Appl. No.: 14/955,210

(22) Filed: Dec. 1, 2015

Related U.S. Application Data
(60) Provisional application No. 62/091,787, filed on Dec. 15, 2014.

(51) Int. Cl. G01N 33/22; G01N 30/68; G01N 30/64; G01N 30/72

(52) U.S. Cl. CPC G01N 33/225; G01N 30/7206; G01N 30/68; G01N 30/64; G01N 2030/025

(57) ABSTRACT

Methods for determining suitability of a fuel are disclosed. In particular the methods include analyzing the fuel for one or more compounds derived from styrene and rejecting the fuel for use if the amount or ratio of the one or more styrene derived compounds exceeds a predetermined amount. Additionally some methods include determining a first amount of styrene in the fuel; correlating the amount of the one- or more compounds derived from styrene with a second amount of styrene in the fuel and rejecting the fuel for use if the sum of the first and second amounts of styrene exceeds a predetermined amount or an established correlation ratio amount.

### CROSS-REFERENCE TO RELATED APPLICATIONS

This application claims the benefit of provisional U.S. Ser. No. 62/091,787, filed Dec. 15, 2014, the entire contents of which are expressly incorporated by reference herein.

### FIELD OF THE INVENTION

The invention relates to methods of determining suitability of a fuel. The method includes analyzing the fuel for styrene-derived compounds, such as styrene oligomers and/or oligomer by products.

### BACKGROUND OF INVENTION

There is a continuing effort to utilize low value fuels in ever more demanding environments. Low value fuels include heavier fractions of crude oil, typically those heavier than gasoline factions or naphtha. Such heavy fuels, however, are prone to sludge formation and fuel filter clogging in use. Sludge formation and/or filter plugging, particularly when used as marine fuels, has a significant impact on the economic viability of such fuels.

Sludge formation and filter clogging have been linked to the presence of styrene in commercial fuels. Thus, testing methods have been developed to determine the amount of styrene in commercial fuels so that their suitability may be ascertained prior to use. It had been believed that acceptable performance may be achieved provided the level of styrene in the fuel is about 2000 ppm or less. Nevertheless, significant cases of sludge formation and/or filter fouling have been observed in fuels wherein little or no styrene was detected by these methods. Likewise, some fuels having relatively high styrene levels seem to provide acceptable performance. Thus, a method of testing heavy fuels that better predicts their suitability for use would be useful.

### SUMMARY OF INVENTION

It has been found that one or more of the above needs may be provided by a testing method that accounts for the likelihood that styrene in the fuel has been converted to styrene-derived compounds, particularly styrene oligomers and or oligomer isomers.

Thus, in one aspect embodiments of the invention provide a method for determining suitability of a fuel, the method comprising: (a) determining a first amount of styrene in the fuel; (b) determining an amount of one or more styrene-derived compounds in the fuel; and (c) rejecting the fuel for use if the sum of the first and second amounts of styrene exceed a predetermined amount or an established correlation ratio amount.

In another aspect, embodiments of the invention provide a method for determining suitability of a fuel, the method comprising: (a) subjecting the fuel to simulated use; (b) determining an amount of one or more styrene-derived compounds in the fuel; and (c) rejecting the fuel for if the amount of the one or more styrene-derived compounds in the fuel exceeds a predetermined amount or an established correlation ratio amount.

In yet another aspect, embodiments of the invention provide a method for determining suitability of a fuel, the method comprising: (a) determining a first amount of styrene in the fuel; (b) determining an amount of one or more styrene-derived compounds in the fuel; (c) correlating the amount of the one or more styrene-derived compounds with a second amount of styrene; and (d) rejecting the fuel for use if a total amount based on the first and second amounts of styrene exceeds a predetermined amount or an established correlation ratio amount.

### BRIEF DESCRIPTION OF THE DRAWINGS

FIG. 1 illustrates a GC/MS Total Ion Chromatogram (TIC) of the reaction products from the styrene polymerization of Example 5.

FIG. 2 illustrates a reconstructed GC/MS for ion m/z=207 chromatogram of the total ion chromatogram (TIC) for the styrene polymerization of Example 5

FIG. 3 illustrates a GC/MS (TIC) of Fuel 1.

FIG. 4 illustrates GC/MS (TIC) mass spectrum of Example 10.

FIG. 5 illustrates mass spectrum extracted ion chromatographs of the TIC in FIG. 4 for (a) ion m/z=104; (b) m/z=91; and (c) ion m/z=207.

FIG. 6 is an expansion of FIG. 5c for the stable trimer region of mass m/z=207.

### DETAILED DESCRIPTION OF THE PREFERRED EMBODIMENTS

This invention relates to a method to determine the suitability of a fuel. It has been found that some fuels that appear to have acceptably low styrene content according to current test methods are still not suitable for use. It is believed that such fuels may pass the testing protocol because problematic styrene may have reacted during transport or storage prior to testing. Thus, the methods described herein test for compounds that may be derived from the reaction of styrene, particularly styrene oligomers and/or oligomer isomers.

As used herein, the term “styrene-derived compounds” means compounds that are reaction or degradation products of styrene monomers, e.g. styrene and methylstyrene. Particular styrene-derived compounds may be a styrene oligomer or stable non-reactive oligomer isomers that are a by-products of a styrene polymerization, e.g., 1e-Phenyl-4e-(10-phenylethyl) tetrahydronaphthalene, 1a-Phenyl-4e-(10-phenylethyl) tetrahydronaphthalene, 1a-Phenyl-4a-(10-phenylethyl) tetrahydronaphthalene, 1e-Phenyl-4a-(10-phenylethyl) tetrahydronaphthalene. Some styrene-derived compounds, including oligomers and oligomer isomers have a molecular weight of about 200.0 to about 2000.0 g/mol, about 200.0 to about 1000.0 g/mol, or about 200.0 to about 500.0 g/mol, e.g. styrene dimer, styrene trimer, styrene tetramer, etc., and isomers thereof. The terms styrene oligomer, styrene dimer, styrene tetramer, styrene tetramer, and styrene oligomer isomer etc. are intended to include methylstyrene analogues. Some styrene-derived compounds are characterized by a peak in a gas chromatographic chromatogram or particularly the GC/MS mass spectrum, of 50.0 to 225 m/z, particularly 85.0 to 95.0 m/z, 100.0 to 110.0 m/z, and 200.0 to 215.0 m/z, wherein “m” is the mass of the observed ion and “z” the charge of the observed ion. For example, styrene trimer and stable trimer isomers (i.e., 1e-Phenyl-4e-(10-phenylethyl) tetrahydronaphthalene, 1a-Phenyl-4e-(10-phenylethyl) tetrahydronaphthalene, 1a-Phenyl-4a-(10-phenylethyl) tetrahydronaphthalene, and 1e-Phenyl-4a-(10-phenylethyl) tetrahydronaphthalene) are characterized by peaks at 91 m/z and 207 m/z.

As used herein the units of parts per million are expressed as “ppm” based on weight (wt/wt)

The type of fuel tested is not critical. Thus, the fuel may have one or more of the following properties.

In any embodiment, the fuel may have a pour point (as measured by ASTM D 97) of ≦about 50.0° C.; e.g., ≦about 40.0° C., ≦about 30.0° C., ≦about 20.0° C., ≦about 10.0° C., ≦about 5.0° C. Additionally or alternatively, the pour point may be ≧about 0° C.; e.g., ≧about 0° C., ≧about 5.0° C., ≧about 10.0° C., ≧about 20.0° C., ≧about 30.0° C., ≧about 40.0° C., ≧about 45.0° C. Exemplary ranges for the pour point of a fuel include about 0 to about 50.0° C., about 0 to about 40.0° C., about 0 to about 30.0° C., about 0 to about 20.0° C., about 0 to about 10.0° C., about 0 to about 5.0° C., about 5.0 to about 50.0° C., about 5.0 to about 40.0° C., about 5.0 to about 30.0° C., about 5.0 to about 20.0° C., about 5.0 to about 10.0° C., about 10.0 to about 50.0° C., about 10.0 to about 40.0° C., about 10.0 to about 30.0° C., about 10.0 to about 20.0° C., about 20 to about 50.0° C., about 20.0 to about 40.0° C., about 20.0 to about 30.0° C., about 30.0 to about 50.0° C., about 30.0 to about 40.0° C., or about 40.0 to about 50.0° C.

In any embodiment, the fuel may have a viscosity at 50° C. (measured by ASTM D 445) of ≦about 1000 centistokes (cSt); e.g., ≦about 750 cSt, ≦about 500 cSt, ≦about 400 cSt, ≦about 300 cSt, ≦about 100 cSt, ≦about 50 cSt, ≦about 25 cSt. Additionally or alternatively, the viscosity of the fuel at 50° C. may be ≧about 20 cSt, ≧about 50 cSt, ≧about 100 cSt, ≧about 200 cSt, ≧about 300 cSt, ≧about 400 cSt, ≧about 500 cSt, or ≧about 750 cSt. Exemplary fuels may have a kinematic viscosity at 50° C. of about 20 to about 1000 cSt, about 20 to about 750 cSt, about 20 to about 500 cSt, about 20 to about 400 cSt, about 20 to about 300 cSt, about 20 to about 200 cSt, about 20 to about 100 cSt, about 20 to about 50 cSt, about 20 to about 25 cSt, 50 to about 750 cSt, about 50 to about 500 cSt, about 50 to about 400 cSt, about 50 to about 300 cSt, about 50 to about 200 cSt, about 50 to about 100 cSt, about 100 to about 500 cSt, about 100 to about 400 cSt, about 100 to about 300 cSt, about 100 to about 200 cSt, about 200 to about 400 cSt, about 200 to about 300 cSt, or about 300 cSt.

In any embodiment, the fuel may have a density (according to ASTM D4052-11) at 15° C. of ≦about 1.02 g/cm³ e.g., ≦about 1.00 g/cm³, ≦about 0.990 g/cm³, ≦about 0.980 g/cm³, ≦about 0.970 g/cm³, ≦about 0.960 g/cm³, ≦about 950 g/cm³. Additionally or alternatively, the density may be ≧0.950 g/cm³, ≧0.960 g/cm³, ≧0.970 g/cm³, ≧0.980 g/cm³, ≧0.990 g/cm³, or ≧1.00 g/cm³. Exemplary fuels may have a density of about 0.950 to about 1.02 g/cm³, about 0.950 to about 1.00 g/cm³, about 0.950 to about 0.990 g/cm³, about 0.950 to about 0.980 g/cm³, about 0.950 to about 0.970 g/cm³, about 0.950 to about 0.960 g/cm³, about 0.960 to about 1.00 g/cm³, about 0.960 to about 0.990 g/cm³, about 0.960 to about 0.980 g/cm³, about 0.960 to about 0.970 g/cm³, about 0.970 to about 1.00 g/cm³, about 0.970 to about 0.990 g/cm³, about 0.980 to about 1.00 g/cm³, about 0.990 to about 1.00 g/cm³, or about 0.990 g/cm³.

The methods described herein are particularly suitable for heavy fuels including fuel oil, e.g., petroleum distillates such as cutter or fluxes stocks as well as residue (i.e., resid). Exemplary fuel oils include vacuum gas oil, atmospheric gas oil, heavy fuel oil, furnace fuel oil, Number 1 fuel oil (i.e., coal oil, stove oil and range oil), Number 2 and 3 fuel oil (e.g., Bunker A fuel, home heating oil, diesel fuel, and light gas oil; Number 4 fuel oil (e.g. heating oil from heavy gas oil; Number 5 fuel oil (e.g., Bunker B fuel, typically from heavy gas oil or a blend of residual oil and Number 2 Fuel Oil; Number 6 fuel oil is a high-viscosity residual oil requiring preheating to 104-127° C. (e.g., Bunker C fuel). Number 5 and 6 fuel are sometimes referred to as navy special fuel oil or navy special. The term residue (or resid) means the material remaining after the more cuts of crude oil have been removed by distillation. Another fuel is referred to as Mazut is derived from Russian petroleum sources.

In any embodiment the fuel may be a fuel described by ISO 8217. ISO 8217 discriminates between two fuel types, RMK and RMG. RMG grade generally being delivered at 380 centistokes or less, and RMK grades at 700 centistokes or less. Other exemplary fuels include DMX marine distillate fuel, DMA, marine distillate fuel, DMB, marine distillate fuel, DMC marine distillate fuel, RMA 30 marine residual fuel, RMB 30 marine residual fuel, RMD 80 marine residual fuel, RME 180 marine residual fuel, RMF 180 marine residual fuel, RMG 380 marine residual fuel, RMH 380 marine residual fuel, RMK 380 marine residual fuel, RMH 700 marine residual fuel, RMK 700 marine residual fuel and mixtures thereof.

In any embodiment, the method may include determining a first amount of styrene in the fuel. The first amount of styrene may be determined by any suitable method. The amount of styrene monomer may be determined for example by gas chromatography according to ASTM D7845. The first amount of styrene is not particularly limited. In particular embodiments, the first amount of styrene determined in this step is ≦about 2000.0 ppm; e.g., ≦about 1500.0 ppm, ≦about 1000.0 ppm, ≦about 500.0 ppm, ≦about 200.0 ppm, or ≦about 100.0 ppm in the fuel. Additionally or alternatively, the first amount of styrene determined in this step may be ≧about 0, ≧about 10.0 ppm, ≧about 50.0 ppm, ≧about 100.0 ppm, ≧about 200.0 ppm, ≧about 500.0 ppm, ≧about 1000.0 ppm, or ≧about 1500.0 ppm. Exemplary first amounts of styrene include about 0 to about 2000.0 ppm, about 0 to about 1500.0 ppm, about 0 to about 1000.0 ppm, about 0 to about 500.0 ppm, about 0 to about 200.0 ppm, about 0 to about 100.0 ppm, about 10.0 to about 2000.0 ppm, about 10 to about 1500.0 ppm, about 10.0 to about 1000.0 ppm, about 10.0 to about 500.0 ppm, about 10.0 to about 200.0 ppm, about 10 to about 100.0 ppm, about 50.0 to about 2000.0 ppm, about 50.0 to about 1500.0 ppm, about 50.0 to about 1000.0 ppm, about 50.0 to about 500.0 ppm, about 50.0 to about 200.0 ppm, about 50.0 to about 100.0 ppm, about 100.0 to about 2000.0 ppm, about 100.0 to about 1000.0 ppm, about 100.0 to about 500.0 ppm, about 100.0 to about 200.0 ppm, about 200.0 to about 2000.0 ppm, about 200.0 to about 1000.0 ppm, about 200.0 to about 500.0 ppm, about 500.0 to 2000.0 ppm, about 500.0 to 1000.0 ppm, or about 1000.0 to 2000.0 ppm. Embodiments where the first amount of styrene is lower may be preferred.

Embodiments of the invention may also include determining an amount of one or more styrene-derived compounds in the fuel. The amount of the one or more styrene-derived compounds may be determined in any manner. Gas chromatography is an exemplary method for determining the amount of the one or more styrene-derived compounds. In particular embodiments the type of gas chromatography may be coupled with a detector. The type of detector is not critical and may be chosen according to convenience. Some suitable detector types include flame ionization detectors (FID), catalytic combustion detectors (CCD), discharge ionization detector (DID), atomic emission detectors (AED), helium ionization detector (HID), infrared detector (IRD), mass spectrometer (MS), photo-ionization detector (PID), pulsed discharge ionization detector (PDD). Of these the combination of gas chromatography with a mass spectrometer (GC/MS) is particularly useful. One of ordinary skill in the art will readily understand how to select conditions suitable for identifying one or more styrene-derived compounds with these methods. GC/MS may also be used to determine the first amount of styrene, which for styrene monomer produces a signal at about 104.0 m/z.

One of ordinary skill in the art will readily understand how to determine the amount of the one or more styrene-derived compounds from the gas chromatograph and selected detection system. For example, a calibration curve may be constructed to determine the detector response to known quantities of particular styrene-derived compounds. The signal strength of the experimental sample is then compared to the calibration curve in order to calculate the amount of styrene-derived compound in the experimental sample. The amount of the one or more styrene-derived compounds is not particularly limited and will depend on the amounts in the fuel being tested. In particular embodiments the amount of the one or more styrene-derived compounds may be ≧about 1000.0 ppm, ≧about 750.0 ppm, ≧about 600.0 ppm, ≧about 500.0 ppm, ≧about 200.0 ppm, ≧about 100.0 ppm, ≧about 50.0 ppm, or ≧about 0 ppm. Additionally or alternatively, the amount of the one- or more styrene-derived compounds may be ≦about 1000.0 ppm, ≦about 750.0 ppm, ≦about 600.0 ppm, ≦about 500.0 ppm, ≦about 500.0 ppm, ≦about 100.0 ppm, ≦about 50.0 ppm or about 0 ppm. Exemplary ranges on the amount of the one or more styrene-derived compounds include about 0 to about 1000.0 ppm, about 0 to about 750.0 ppm, about 0 to about 500.0 ppm, about 0 to about 200.0 ppm, about 0 to about 100.0 ppm, about 0 to about 50.0 ppm, about 50.0 to about 1000.0 ppm, about 50.0 to about 750.0 ppm, about 50.0 to about 500.0 ppm, about 50.0 to about 200.0 ppm, about 50.0 to about 100.0 ppm, about 100.0 to about 1000.0 ppm, about 50.0 to about 750.0 ppm, about 100.0 to about 500.0 ppm, about 100.0 to about 200.0 ppm, about 200.0 to about 1000.0 ppm, about 200.0 to about 750.0 ppm, about 200.0 to about 500.0 ppm, about 500.0 to about 1000.0 ppm, or about 500.0 to about 750.0 ppm.

In any embodiment, the methods according to the invention include rejecting the fuel for use if a total amount based on the amount of the one or more styrene-derived compounds and the first amount of styrene exceeds a predetermined amount. The predetermined amount is typically determined by a maximum of the sum of the amount of the one or more styrene-derived compounds and the first amount of styrene that permits acceptable function of the fuel. Where a fuel having less clogging tendency is desired, the predetermined amount may be less than in applications having a higher tolerance. In particular embodiments, the predetermined amount may be ≦about 2000.0 ppm, e.g., ≦about 1750.0 ppm, ≦about 1500.0 ppm, ≦about 1250.0 ppm,≦about 1000.0 ppm, ≦about 750.0 ppm, or ≦about 500.0 ppm. Exemplary predetermined amounts include 0 to about 2000.0 ppm, 0 to about 1750.0 ppm, 0 to about 1500.0 ppm, 0 to about 1250.0 ppm, 0 to about 750.0 ppm, 0 to about 600 ppm, 0 to about 500 ppm, about 500.0 to about 2000.0 ppm, about 500.0 to about 1750.0 ppm, about 500.0 to about 1500.0 ppm, about 500.0 to about 1250.0 ppm, about 500.0 to about 750.0 ppm, about 500.0 to about 600 ppm, about 600.0 to about 2000.0 ppm, about 600.0 to about 1750.0 ppm, about 600.0 to about 1500.0 ppm, about 600.0 to about 1250.0 ppm, about 600.0 to about 750.0 ppm, about 750.0 to about 2000.0 ppm, about 750.0 to about 1750.0 ppm, about 750.0 to about 1500.0 ppm, about 750.0 to about 1250.0 ppm, about 1000.0 to about 2000.0 ppm, about 1000.0 to about 1750.0 ppm, about 1000.0 to about 1500.0 ppm, about 1000.0 to about 1250.0 ppm, about 1250.0 to about 2000.0 ppm, about 1250.0 to about 1750.0 ppm, about 1250.0 to about 1500.0 ppm, about 1500.0 to about 2000.0 ppm, about 1500.0 to about 1750.0 ppm, and about 1750.0 to 2000.0 ppm. The total amount of the one or more styrene-derived compounds and the first amount of styrene is typically the sum of the individual amounts of the one or more styrene-derived compounds and the first amount of styrene. But any convenient manipulation of the individual amounts may be used, provided however, that an equivalence manipulation is applied to the predetermined value. Particularly in embodiments described below where the fuel is subjected to simulated use prior to testing, the predetermined amount may be lower. In such cases, it is believed that the styrene monomers are converted to styrene oligomers. Thus, as described above a lower measured value for the one or more styrene-derived compounds corresponds to relatively higher styrene content. Thus, the predetermined amount may be set accordingly at any value described above.

For example, in particular embodiments of the invention the amount of the one or more styrene-derived compounds may be correlated with a second amount of styrene corresponding thereto. Doing so may be beneficial to understand the styrene content of the fuel before the reactive styrene is converted to the one or more styrene-derived compounds. Correlating the amount of the one or more styrene-derived compounds with a second amount of styrene may be accomplished by any suitable means. One of ordinary skill in the art will recognize that based on the identity of the styrene monomer and the styrene-derived compounds, the amount of styrene needed to form the styrene-derived compound can be readily calculated. For example, where the styrene-derived compound is a styrene trimer, the number of styrene monomers needed to form one trimer molecule is three. Thus, the amount (or concentration) of styrene that correlates to the amount of the one or more styrene-derived compounds is 3 times the measured amount of the one or more styrene-derived compounds.

It may be convenient to assist the conversion of the styrene monomer in the fuel prior to testing. Thus, additionally or alternatively, embodiments of the invention may include subjecting the fuel to actual or simulated use prior to testing. Such testing may convert all or a portion of the styrene monomer to styrene-derived compounds. While conversion of essentially all styrene monomers to styrene-derived compounds may be preferred, methods wherein conditions are used to convert a portion of the styrene monomer from which the total amount of the styrene and/or styrene-derived compounds could be estimated. Such embodiments may be useful particularly where the conversion of styrene monomer is not particular fast. In particular embodiments, subjecting the fuel to simulated use includes mixing the fuel at a temperature and for a time sufficient for at least a portion, preferably substantially all, of the styrene monomer in the fuel to form the one or more styrene-derived compounds. Of course, subjecting the fuel to simulated use may include the use of temperature profiles, i.e., subjecting the fuel to a two or more of the following temperatures for one or more of the following predetermined times.

While any convenient temperature may be used, temperatures≧about 80.0° C. are typically useful, e.g. ≧about 100.0° C., ≧about 125.0° C., ≧about 150.0° C., ≧about 175.0° C., ≧about 200.0° C., ≧about 225.0° C., ≧about 250.0° C., ≧about 275.0° C. Additionally or alternatively, the temperature may be ≦about 275.0° C., ≦about 250.0° C., ≦about 225.0° C., ≦about 200.0° C., ≦about 175.0° C., ≦about 150.0° C., ≦about 125.0° C., ≦about 100.0° C. Exemplary ranges of temperatures include about 80.0 to about 275.0° C., 80.0 to about 250.0° C., 80.0 to about 225.0° C., 80.0 to about 200.0° C., 80.0 to about 175.0° C., 80.0 to about 150.0° C., 80.0 to about 125.0° C., 80.0 to about 100.0° C., about 100.0 to about 275.0° C., 100.0 to about 100.0° C., 100.0 to about 225.0° C., 100.0 to about 200.0° C., 100.0 to about 175.0° C., 100.0 to about 150.0° C., 100.0 to about 125.0° C., about 125.0 to about 275.0° C., 125.0 to about 250.0° C., 125.0 to about 225.0° C., 125.0 to about 200.0° C., 125.0 to about 175.0° C., 125.0 to about 150.0° C., about 150.0 to about 275.0° C., 150.0 to about 250.0° C., 150.0 to about 225.0° C., 150.0 to about 200.0° C., 150.0 to about 175.0° C., about 175.0 to about 275.0° C., 175.0 to about 250.0° C., 175.0 to about 225.0° C., 175.0 to about 200.0° C., about 200.0 to about 275.0° C., 200.0 to about 250.0° C., 200.0 to about 225.0° C., about 225.0 to about 275.0° C., 225.0 to about 250.0° C., and about 250.0 to about 275.0° C.

Simulated use may be conducted for any convenient predetermined time period depending on any number of factors, e.g., temperature etc. Typically however, a predetermined time of ≧about 0.5 hours, e.g., ≧about 0.5 hours, ≧about 1.0 hours, ≧about 2.0 hours, ≧about 5.0 hours, ≧about 10.0 hours≧about 25.0 hours≧about 50.0 hours, ≧about 75.0 hours, ≧about 100.0 hours. Additionally or alternatively, the predetermined period of time may be ≦about 100.0 hours, e.g., ≦about 75.0 hours, ≦about 50.0 hours, ≦about 25.0 hours, ≦about 10.0 hours, ≦about 2.0 hours, ≦about 1.0 hours. Exemplary time periods include about 0.5 to about 100.0 hours, about 0.5 to about 75.0 hours, about 0.5 to about 50.0 hours, about 0.5 to about 25.0 hours, about 0.5 to about 10.0 hours, about 0.5 to about 5.0 hours, about 0.5 to about 2.0 hours, about 0.5 to about 1.0 hours, about 1.0 to about 100.0 hours, about 1.0 to about 75.0 hours, about 1.0 to about 50.0 hours, about 1.0 to about 25.0 hours, about 1.0 to about 10.0 hours, about 1.0 to about 5.0 hours, about 1.0 to about 2.0 hours, about 2.0 to about 100.0 hours, about 2.0 to about 75.0 hours, about 2.0 to about 50.0 hours, about 2.0 to about 25.0 hours, about 2.0 to about 10.0 hours, about 2.0 to about 5.0 hours, about 5.0 to about 100.0 hours, about 5.0 to about 75.0 hours, about 5.0 to about 50.0 hours, about 5.0 to about 25.0 hours, about 5.0 to about 10.0 hours, about 10.0 to about 100.0 hours, about 10.0 to about 75.0 hours, about 10.0 to about 50.0 hours, about 10.0 to about 25.0 hours, about 25.0 to about 100.0 hours, about 25.0 to about 75.0 hours, about 25 to about 50.0 hours, about 50.0 to about 100.0 hours, about 50.0 to about 75.0 hours, and about 75.0 to about 100.0 hours.

Mixing may be accomplished by any suitable means. In particular embodiments, mixing may be accomplished by a recirculation unit, e.g. a Hot Liquid Processing Unit (HLPS).

### ADDITIONAL EMBODIMENTS

### Embodiment 1

A method for determining suitability of a fuel, the method comprising: (a) determining a first amount of styrene in the fuel; (b) determining an amount of one or more styrene-derived compounds in the fuel; and (c) rejecting the fuel for use if a total amount based on the amount of the one or more styrene-derived compounds and the first amount of styrene exceeds a predetermined amount or an established correlation ratio amount.

### Embodiment 2

A method for determining suitability of a fuel, the method comprising: (a) subjecting the fuel to simulated use; (b) determining an amount of one or more styrene-derived compounds in the fuel; and (c) rejecting the fuel for if the amount of the one or more styrene-derived compounds in the fuel exceeds a predetermined amount or an established correlation ratio amount.

### Embodiment 3

A method for determining suitability of a fuel, the method comprising: (a) determining a first amount of styrene in the fuel; (b) determining an amount of one or more styrene-derived compounds in the fuel; (c) correlating the amount of the one or more styrene-derived compounds with a second amount of styrene; and (d) rejecting the fuel for use if a total amount based on the first and second amounts of styrene exceeds a predetermined amount or an established correlation ratio amount.

### Embodiment 4

The method according any of Embodiments 1-3, wherein the fuel is selected from the group consisting of resid, bunker fuel, home heating oil, vacuum gas oil, atmospheric gas oil, heavy fuel oil, furnace fuel oil and mixtures thereof.

### Embodiment 5

The method according any of Embodiments 1-3, wherein the fuel is selected from the group consisting of marine gas oil, marine diesel oil, intermediate fuel oil, marine fuel oil, marine distillate fuel, marine residual fuel, and mixtures thereof.

### Embodiment 6

The method according to Embodiment 5, wherein the fuel is selected from DMX marine distillate fuel, DMA, marine distillate fuel, DMB, marine distillate fuel, DMC marine distillate fuel, RMA 30 marine residual fuel, RMB 30 marine residual fuel, RMD 80 marine residual fuel, RME 180 marine residual fuel, RMF 180 marine residual fuel, RMG 380 marine residual fuel, RMH 380 marine residual fuel, RMK 380 marine residual fuel, RMH 700 marine residual fuel, RMK 700 marine residual fuel and mixtures thereof.

### Embodiment 7

The method according to any of Embodiments 1 and 3-6, wherein the first amount of styrene determined in step (a) is ≦about 2000.0 ppm, ≦about 1500.0 ppm, ≦about 1000.0 ppm, ≦about 500.0 ppm, ≦about 200.0 ppm, or ≦about 100.0 ppm in the fuel.

### Embodiment 8

The method according to any of Embodiments 1-7, wherein the predetermined amount is ≦about 2000.0 ppm, ≦about 1750.0 ppm, ≦about 1500.0 ppm, ≦about 1250.0 ppm, ≦about 1000.0 ppm, ≦about 750.0 ppm≦about 500.0 ppm.

### Embodiment 9

The method according to any of Embodiments 1-8, wherein the one or more styrene-derived compounds have a molecular weight of about 200.0 to about 2000.0 g/mol, about 200.0 to about 1000.0 g/mol, or about 200.0 to about 500.0 g/mol.

### Embodiment 10

The method according to any of Embodiments 1-9, wherein the one or more styrene-derived compounds comprise a styrene trimer or styrene stable trimer isomer.

### Embodiment 11

The method according to any of Embodiments 1-10, wherein the one or more styrene-derived compounds are selected form the group consisting of 1e-Phenyl-4e-(10-phenylethyl) tetrahydronaphthalene, 1a-Phenyl-4e-(10-phenylethyl) tetrahydronaphthalene, 1a-Phenyl-4a-(10-phenylethyl) tetrahydronaphthalene, 1e-Phenyl-4a-(10-phenylethyl) tetrahydronaphthalene and combinations or mixtures thereof.

### Embodiment 12

The method according to any of Embodiments 1-11, wherein determining the amount of one or more styrene-derived compounds includes analyzing the resulting mixture by a method comprising gas chromatography.

### Embodiment 13

The method according to any of Embodiments 1-12, wherein determining the amount of one or more styrene-derived compounds includes analyzing the resulting mixture by GC/MS, GC/FID, GC/PID, and gas chromatography.

### Embodiment 14

The method according to Embodiment 13, wherein the GC/MS spectrum possess a signal in the range from 50.0 to 225 m/z, particularly 85.0 to 95.0 m/z, 100.0 to 110.0 m/z, and 200.0 to 215.0 m/z, in the spectrum.

### Embodiment 15

The method according to any of Embodiments 2 and 4-14, wherein subjecting the fuel to simulated use includes mixing the marine fuel mixture at a temperature and time sufficient to cause at least a portion of any styrene therein to form styrene-derived compounds (e.g., about 80° C. to about 275° C., about 100° C. to about 250° C., or about 125° C. to about 225° C., for a predetermined period of time, such as from about 0.5 to about 100.0 hours, about 1.0 to about 75.0 hours, about 2.0 to about 50.0 hours, or about 5.0 to about 25 hours.

### Embodiment 16

The method according to any of Embodiments 2 and 4-15, wherein the predetermined amount is ≦about 1000.0 ppm, ≧about 750.0 ppm≧about 600.0 ppm≧about 500.0 ppm.

### EXAMPLES

### Examples 1-4

In Examples 1-4, commercial fuels were tested by GC/MS, and the relative amounts of the styrene were determined. In particular, the amount of styrene was determined from the peak at 104 m/z. These values were compared to the observed performance of the fuels. These results are tabulated in Table 1.

TABLE 1
Comparison of Commercial Fuel Samples

| | | Styrene | |
|---|---|---|---|
| Example | Fuel Sample | (ppm) | Problematic |
| 1 | Fuel 1 | ~2190 | YES |
| 2 | Fuel 2 | ~326 | NO |
| 3 | Fuel 3 | ~2300 | NO |
| 4 | Fuel 4 | ~134 | NO |

From Examples 1-4, it can be seen that Fuels 1 and 3 each exhibited an amount exceeding a typical ˜2000 ppm limit on styrene content. Fuels 2 and 4 having relatively low styrene content significantly below the ˜2000 ppm threshold were not problematic. While Fuel 1 proved to be problematic in use due to sludge formation, Fuel 3 showed acceptable performance despite its higher styrene content.

### Example 5

Example 5 investigated the reactivity of styrene under simulated use conditions. Styrene (˜5.0 mL) was heated at ˜275° C. for ˜24 hours in a ˜75 mL high pressure steel vessel. The resulting mixture was analyzed by GC/MS total ion chromatogram (TIC) and selective extracted mass spectrum ions m/z. The resulting chromatograms were compared to that of styrene monomer and the stable trimer isomers including isomers of 1-phenyl-4-(1′-phenylethyl)tetralin, i.e., 1e-Phenyl-4e-(10-phenyl ethyl) tetrahydronaphthalene, 1a-Phenyl-4e-(10-phenylethyl) tetrahydronaphthalene, 1a-Phenyl-4a-(10-phenylethyl) tetrahydronaphthalene, and 1e-Phenyl-4a-(10-phenylethyl) tetrahydronaphthalene, the structure of which is shown below.

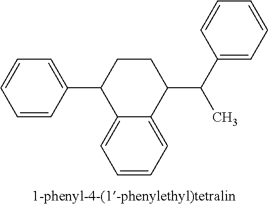 [SMILES: CC(c1ccccc1)C1CCC(c2ccccc2)c2ccccc21]

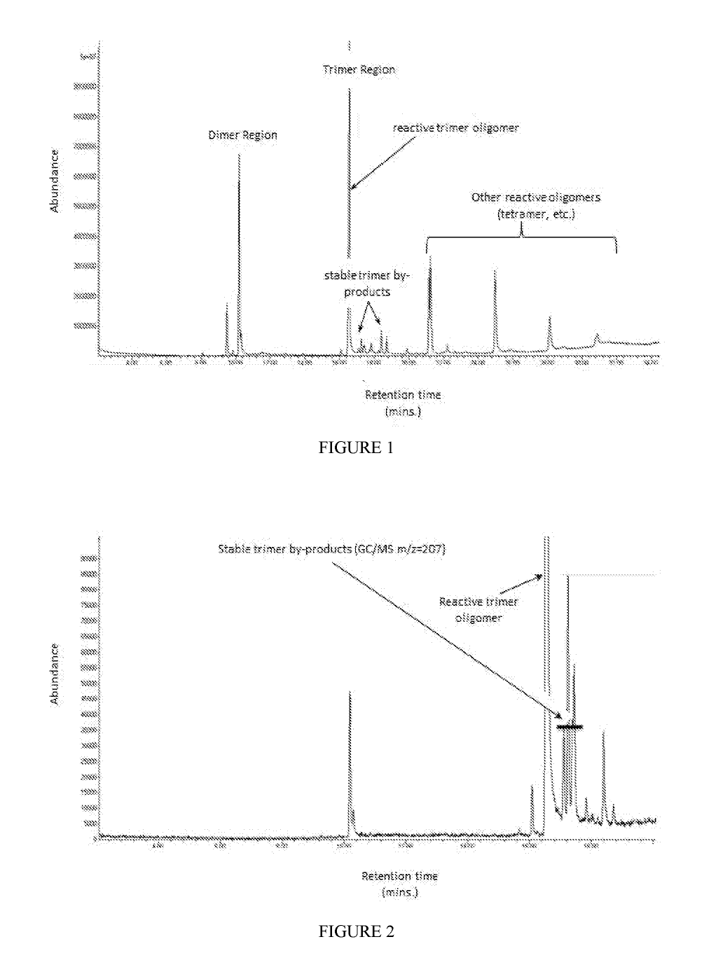

Standards of these styrene stable trimer compounds are available from Wako Chemicals, Ltd. (Japan) and can be used to establish the GC/MS operating conditions for compound elution times and identify from their mass spectra. Alternatively, such isomers may be prepared by heating a styrene and examining the reaction products. As FIG. 1 shows, the styrene monomer reacted completely and was not detected at the expected low elution retention times. FIG. 1 also shows that styrene oligomers, e.g., dimers, trimers, tetramers etc., were present. In addition, smaller amounts of the stable trimer isomers were indicated. FIG. 2 is an expanded view of FIG. 1, but using selective ion m/z 207 focusing on the peaks observed retention times in region from 0 to 20 min, that showed both the reactive trimer as a major compound plus smaller amounts of the stable styrene trimers. FIG. 2 showed that two of the four stable m/z 207 stable isomers appeared to co-elute, based on using the Wako Chemicals pure standards used to identify these trimers and that the reactive trimer oligomer was present in much higher concentration.

### Example 6

To determine if the performance of Fuel 1 may be linked to the oligomerization of styrene, Example 6 examined the GC/MS spectrum of Fuel 1. Thus, the GC/MS (TIC) spectrum of Fuel 1 is depicted in FIG. 3. Included in the spectrum was a cluster of peaks near a retention time of ˜17 mins that corresponded to the styrene stable non-reactive trimer isomers, the mass spectra of which each contained ions having an m/z of 207. Without wishing to be held to any particular theory, it is believed that these stable trimer isomers were indicative that styrene oligomerization had occurred and the probability that the more reactive trimer oligomer had been present was converted into problematic higher molecular weight compounds. A ratio of the amount of these isomers to the amount of styrene in Fuel was found to be about ˜14.0.

### Examples 7-9

Example 6 was substantially repeated except that Fuel 1 was replaced by Fuels 2, 3, and 4, respectively. The relative amounts of styrene and the ratio of styrene (m/z=104) to the stable trimer isomers associated with an m/z value of 207 were reported in Table 2.

TABLE 2
Comparison of Commercial Fuel Samples

| | | Styrene | Ratio of Peaks | |
|---|---|---|---|---|
| Example | Fuel Sample | (ppm, w/w) | 207/104 | Problematic |
| 6 | Fuel 1 | ~2190 | ~14 | YES |
| 7 | Fuel 2 | ~326 | 0 | NO |
| 8 | Fuel 3 | ~2300 | 0 | NO |
| 9 | Fuel 4 | ~134 | 0 | NO |

Inspection of Table 2 shows that problematic Fuel 1 not only possessed a high amount of styrene, but also a relatively large amount of stable trimer isomers, where the ratio of the peaks indicated that a styrene polymerization had occurred, which most likely produced higher molecular weight oligomers, which, under certain conditions, can lead to sludge formation on use of the fuel. It should be noted that the stable 207 m/z trimers are usually produced in relatively much smaller amount than the higher molecular weight styrene reactive oligomers, which continue possibly to sludge. Thus, the detection of the stable isomers in the fuel even at lower concentration levels may point to a much higher concentration of higher molecular weight reactive styrene oligomers being present. In the latter sense, the stable trimers become a ‘marker’ that significant styrene polymerization has occurred. To the contrary, Fuel 3, which also had high styrene content but performed acceptably, did not exhibit the peaks indicative of the trimer isomers.

### Examples 10-13

In Examples 10-13, the reaction products of styrene were examined in a second commercial flux having most of its components boiling in diesel range of an initial boiling point (IBP) of ˜135° C. (˜275° F.) and a final boiling point (FBP) of about ˜425° C. (˜800° F.). The initial styrene content of the flux was below the detection limit. Thus, the flux was spiked with varying concentrations of styrene ranging from ˜9% to ˜0.7% (m/m), as shown in Table 3. Approximately 50 g of the spiked flux was heated statically at ˜275° C. for total of ˜30 hours in the ˜75 cc high pressure steel vessels. The flux was also sampled once after ˜6 hours of heating. The heated flux was analyzed by GC/MS to determine the presence of products by monitoring for the presence of reactive styrene oligomers by monitoring for ions with an m/z of 207, 91, and 104.

TABLE 3

| | | Amount (g) of | | |
|---|---|---|---|---|
| | | Styrene | | |
| | Initial styrene | added to | Temperature | final styrene |
| Example | content (ppm) | 50 g of flux | (° C.) | content (%) |
| 10 | <50 ppm | 5 | 275 | 9.1 |
| 11 | <50 ppm | 2.50 | 275 | 4.8 |
| 12 | <50 ppm | 1.00 | 275 | 2.0 |
| 13 | <50 ppm | 0.150 | 275 | 0.7 |

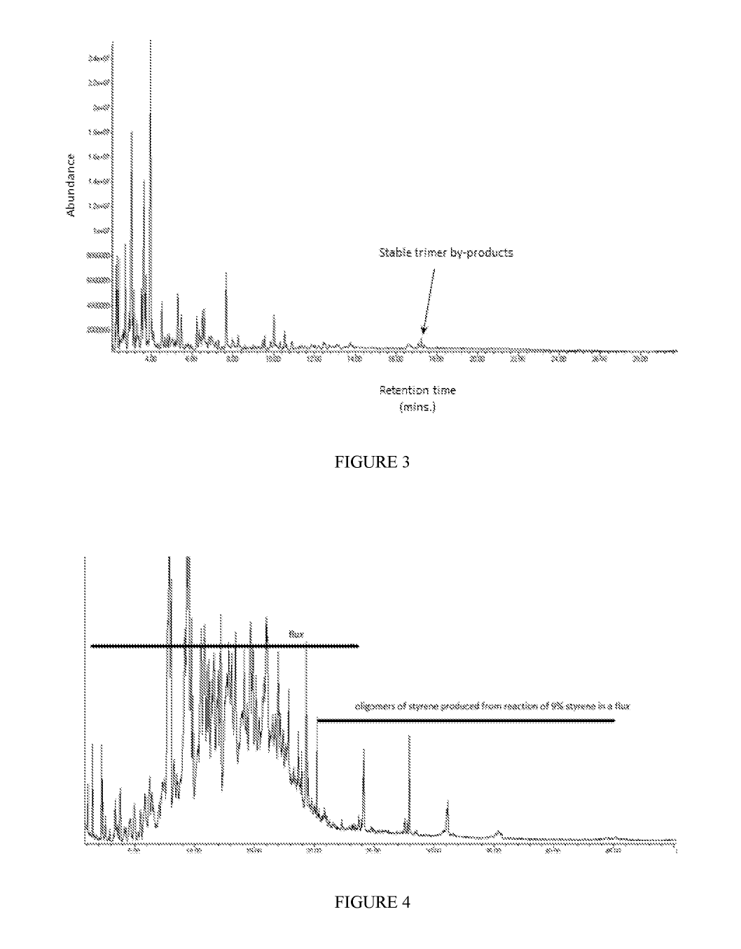

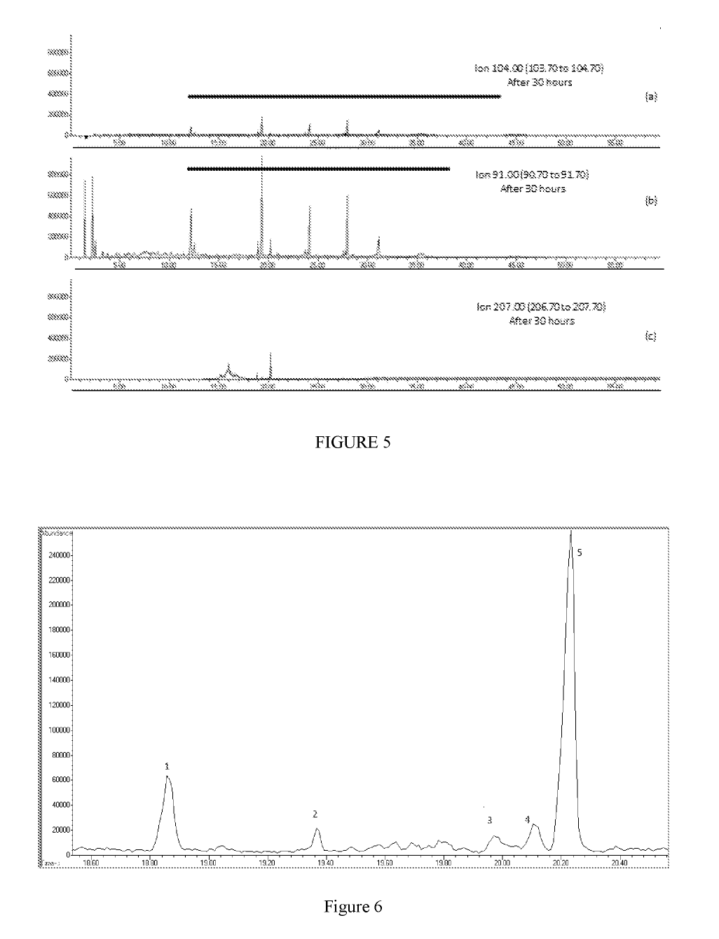

FIG. 4 shows the GC/MS TIC chromatogram of Example 10 after heating for ˜30 hours. In the interference-free regions of the flux components, the styrene oligomers were indicated. FIG. 5(a-c) represented the extracted ions m/z 104, 91, and 207 of the total ion chromatogram of in FIG. 4, which can selectively locate these oligomers even if co-eluting with other flux hydrocarbons and hetero-atom compounds. Selectively, the three mass ions showed the presence of the styrene oligomers plus the stable trimer isomer compounds containing the m/z=207 [5(c)] mass ions in the GC/MS retention or elution times of ˜17-20 minutes. FIG. 5(a) indicated that no detectable amount of styrene monomer was detected by mass m/z=104 after ˜30 hours of heating. A similar chromatogram (not shown) after ˜6 hours of heating indicated that some of the monomer remained. FIG. 6 is an expansion of FIG. 5c for the stable trimer region of mass m/z=207. FIG. 6 indicated that several isomers of the stable trimers were formed whose mass spectra all contained the m/z 207 ions. Heating of the styrene in the relatively uncomplicated flux produced several trimer isomers in addition to the reactive trimer.

Analyses of Examples 11-13, having ˜4.8%, ˜2.0%, and ˜0.7% (m/m) styrene, respectively, showed substantially the same result. Detection of the oligomers and m/z=207 was observed for Examples 11 and 12, but not for Example 13, indicating that the ‘detection limit’ of detectability for such stable trimers components was equivalent to an original monomer concentration of between ˜2.0% and ˜0.7% styrene in the flux. At the ˜0.7% (˜7000 ppm) styrene content level, all of the styrene appeared to have reacted, as the residual monomer was not detected; the latter suggested that, even for a ˜0.7% styrene, approximately 7000 ppm of oligomers may have been created in the flux but remain undetected. In large quantities of fuels, such even such low concentrations in the fuel could cause sludge if used.

All documents described herein are incorporated by reference herein for purposes of all jurisdictions where such practice is allowed, including any priority documents and/or testing procedures to the extent they are not inconsistent with this text, provided however that any priority document not named in the initially filed application or filing documents is NOT incorporated by reference herein. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including” for purposes of Australian law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

What is claimed is:

1. A method for determining suitability of a fuel, the method comprising:
(a) determining a first amount of styrene in the fuel;
(b) determining an amount of one or more styrene-derived compounds in the fuel; and
(c) rejecting the fuel for use if a total amount based on the amount of the one or more styrene-derived compounds and the first amount of styrene exceeds a predetermined amount or an established correlation ratio amount.

2. The method of claim 1, wherein the fuel is selected from the group consisting of resid, bunker fuel, home heating oil, cutter or flux used as diluent of heavy oils, vacuum gas oil, atmospheric gas oil, heavy fuel oil, furnace fuel oil and mixtures thereof.

3. The method of claim 1, wherein the fuel is selected from the group consisting of marine gas oil, marine diesel oil, intermediate fuel oil, marine fuel oil, marine distillate fuel, marine residual fuel, and mixtures thereof.

4. The method of claim 3, wherein the fuel is selected from DMX marine distillate fuel, DMA, marine distillate fuel, DMB, marine distillate fuel, DMC marine distillate fuel, RMA 30 marine residual fuel, RMB 30 marine residual fuel, RMD 80 marine residual fuel, RME 180 marine residual fuel, RMF 180 marine residual fuel, RMG 380 marine residual fuel, RMH 380 marine residual fuel, RMK 380 marine residual fuel, RMH 700 marine residual fuel, RMK 700 marine residual fuel and mixtures thereof.

5. The method of claim 1, wherein the first amount of styrene determined in step (a) is ≦about 2000.0 ppm.

6. The method of claim 1, wherein the amount of the one or more styrene-derived compounds in the fuel is ≦about 2000.0 ppm.

7. The method of claim 1, wherein the predetermined amount is about 2000.0 ppm or greater.

8. The method of claim 1, wherein the one or more styrene-derived compounds have a molecular weight of about 200.0 to about 2000.0 g/mol.

9. The method of claim 1, wherein the one or more styrene-derived compounds comprise a styrene oligomers or stable styrene trimer isomer(s).

10. The method of claim 1, wherein the one or more styrene-derived compounds are selected form the group consisting of 1e-Phenyl-4e-(10-phenylethyl) tetrahydronaphthalene, 1a-Phenyl-4e-(10-phenylethyl) tetrahydronaphthalene, 1a-Phenyl-4a-(10-phenylethyl) tetrahydronaphthalene, 1e-Phenyl-4a-(10-phenylethyl) tetrahydronaphthalene and combinations or mixtures thereof.

11. The method of claim 10, wherein determining the amount of one or more styrene-derived compounds includes analyzing the resulting mixture by a method comprising gas chromatography.

12. The method of claim 11, wherein the method comprising gas chromatography is selected from the group consisting of GC/MS, GC/FID, GC/PID, and gas chromatography.

13. The method of claim 11, wherein the GC/MS spectrum possess a signal in the range from 50.0 to 225.0 m/z.

14. A method for determining suitability of a fuel, the method comprising:
(a) subjecting the fuel to simulated use;
(b) determining an amount of one or more styrene-derived compounds in the fuel; and
(c) rejecting the fuel for if the amount of the one or more styrene-derived compounds in the fuel exceeds a predetermined amount or an established correlation ratio amount.

15. The method of claim 14, wherein subjecting the mixture simulated use includes mixing the marine fuel mixture at a temperature sufficient to cause at least a portion of any styrene therein to form styrene-derived compounds.

16. The method of claim 15, wherein the temperature is about 80° C. to about 275° C. for a predetermined period of time.

17. The method of claim 16, wherein the predetermined period of time is from about 0.5 to about 100.0 hours.

18. The method of claim 14, wherein the fuel is selected from the group consisting of resid, bunker fuel, home heating oil, vacuum gas oil, cutter or flux used as diluent of heavy oils, atmospheric gas oil, heavy fuel oil, furnace fuel oil and mixtures thereof.

19. The method of claim 14, wherein the fuel is selected from the group consisting of marine gas oil, marine diesel oil, intermediate fuel oil, marine fuel oil, marine distillate fuel, marine residual fuel, and mixtures thereof.

20. The method of claim 19, wherein the fuel is selected from DMX marine distillate fuel, DMA, marine distillate fuel, DMB, marine distillate fuel, DMC marine distillate fuel, RMA 30 marine residual fuel, RMB 30 marine residual fuel, RMD 80 marine residual fuel, RME 180 marine residual fuel, RMF 180 marine residual fuel, RMG 380 marine residual fuel, RMH 380 marine residual fuel, RMK 380 marine residual fuel, RMH 700 marine residual fuel, RMK 700 marine residual fuel and mixtures thereof.

21. The method of claim 14, wherein the predetermined amount is an amount of styrene-derived compounds is indicative of a styrene content of ≧about 2000.0 ppm in the fuel.

22. The method of claim 14 wherein the predetermined amount is ≦about 600.0 ppm.

23. The method of claim 14, wherein the one or more styrene-derived compounds have a molecular weight of about 200.0 to about 2000.0 g/mol.

24. The method of claim 14, wherein the one or more styrene-derived compounds comprise a styrene trimer or styrene trimer isomer.

25. The method of claim 14, wherein the one or more styrene-derived compounds are selected form the group consisting of 1e-Phenyl-4e-(10-phenylethyl) tetrahydronaphthalene, 1a-Phenyl-4e-(10-phenylethyl) tetrahydronaphthalene, 1a-Phenyl-4a-(10-phenylethyl) tetrahydronaphthalene, 1e-Phenyl-4a-(10-phenylethyl) tetrahydronaphthalene and combinations or mixtures thereof.

26. The method of claim 14, wherein determining the amount of one or more styrene-derived compounds includes analyzing the resulting mixture by a method comprising gas chromatography.

27. The method of claim 26, wherein the method comprising gas chromatography is selected from the group consisting of GC/MS, GC/FID, GC/PID, and gas chromatography.

28. The method of claim 27, wherein the GC/MS spectrum possess a signal in the range from 50.0 to 225 m/z.

29. A method for determining suitability of a fuel, the method comprising:
(a) determining a first amount of styrene in the fuel;
(b) determining an amount of one or more styrene-derived compounds in the fuel;
(c) correlating the amount of the one or more styrene-derived compounds with a second amount of styrene; and
(d) rejecting the fuel for use if a total amount based on the first and second amounts of styrene exceeds a predetermined amount or an established correlation ratio amount.
